(19) United States
(12) Patent Application Publication
(10) Pub. No.: US 2023/0285153 A1
(43) Pub. Date: Sep. 14, 2023

(54) DENTAL MESH FOR COVERING BONE GRAFT MATERIAL

(71) Applicants: Jeff Sung Hwi CHO, Cypress, CA (US); Abraham SoonJong CHO, Cypress, CA (US)

(72) Inventors: Jeff Sung Hwi CHO, Cypress, CA (US); Abraham SoonJong CHO, Cypress, CA (US)

(21) Appl. No.: 17/694,450

(22) Filed: Mar. 14, 2022

(51) Int. Cl. A61F 2/28

(52) U.S. Cl. CPC A61F 2/2846; A61F 2002/285

(57) ABSTRACT

A dental mesh for covering a membrane material and a bone graft material filling the tooth socket includes a mesh and a plurality of attachment needles. The mesh is substantially rectangular, and each needle has an end having a pointed tip and an opposite end connected to the mesh. The attachment needles are curved such that the pointed tips of paired attachment needles face each other or downwardly to respectively penetrate into the opposite gum tissues for securing the mesh to the gum tissues. The attachment needles are connected to strands extending from the mesh. The attachment needles may be “J”-shaped, compound curved, “C”-shaped or “L”-shaped. The mesh further includes a plurality of protrusions, which extend and taper downwardly from the mesh in order to penetrate into the membrane for preventing the membrane from moving.

### FIELD OF THE INVENTION

The present invention relates to a dental mesh for covering a membrane material placed on a bone graft material filling a tooth socket of a patient. More particularly, the invention relates to a dental mesh having a mesh and a plurality of attachment needles constructed to penetrate into gum tissue for securing the mesh in place.

### BACKGROUND OF THE INVENTION

Dental implant surgery is the process where a dental implant is inserted into the jawbone of a patient in order to replace damaged or missing teeth with artificial teeth. Dental implants hold the artificial teeth or bridge in place, and the artificial teeth look and function like natural teeth. A dental implant is typically comprised of a titanium screw, serving as the root of a damaged or missing tooth to support an artificial tooth. The dental implant is inserted into the jawbone and allowed to fuse with the bone over a few months, and then, artificial teeth are attached to the dental implant.

A dental bone graft may be necessary before inserting a dental implant if bone loss has occurred or the jawbone is not strong or thick enough for the implant. During this procedure, bone graft material is placed into a tooth socket to regenerate and rebuild lost jawbone by creating more volume and density to the jawbone. The bone graft material may be an autogenous bone graft, an allograft, a xenograft, an alloplast, or the like.

The dental bone graft procedure includes making an incision in the gum tissue to separate it from the bone, filling the tooth socket or the bone where the bone graft is necessary with the bone graft material, securing the bone graft material using membrane or other material, and sewing up the incision for healing. A membrane is used to cover the bone graft material to prevent the invasion of cells not involved in bone formation. The membrane is placed on the bone graft material in between the gum tissue and the alveolar bone, and the gum tissue is then sutured to secure the membrane in place.

The membrane may be resorbable or non-resorbable. Resorbable membranes include collagen membranes, pericardium membranes, etc. Non-resorbable membranes include polytetrafluoroethylene (PTFE) membranes, titanium membranes, etc. The membrane should have the mechanical strength and stiffness to maintain space for bone growth and prevent collapse of the bone graft site. At the same time, the membrane should be flexible enough to allow size and shape modification. The membrane should prevent passage of unwanted cells and protect the bone graft material from falling out of the bone graft site.

To maintain the membrane in place, the gum tissue is sutured. However, competent suturing skills are hard to acquire and require dedicated training and practice, good understanding of the proper techniques and wound healing process, good hand-eye coordination, etc. Gum tissue is especially soft and sensitive, and vulnerable to damage and irritation. Thus, suturing gum tissue requires specialized suturing skills.

Among many suturing methods, a figure of 8 suture technique is most commonly used. The technique uses criss-cross stitches to quickly close open wounds or finish up oral surgery. While it enables rapid closure, it leaves a significant amount of suture threads inside the tooth socket and the suture is difficult to remove. The suture is incomplete and may result in bone loss.

Improperly placed sutures may result in serious consequences, such as bleeding, infection, pain, and soreness. Even if the suturing is done properly, it is possible for the suture to become loose or break, which may lead to the membrane being moved, slipped, dislocated, or dislodged. Then, the bone graft material may fall out of the bone graft site and the bone may not properly or sufficiently grow. In some cases, additional bone graft surgery may be necessary. After the bone graft surgery, the membrane is exposed and the surgery site is especially vulnerable because the patient has to chew food and clean his/her teeth. Defects and failures of dental bone grafts are not uncommon.

Therefore, to solve the above problems, various embodiments of a dental mesh for covering a membrane placed on the bone graft material are provided, as there is a need for a device that accomplishes these goals. This invention is directed to solve these problems and satisfy the long-felt need.

### SUMMARY OF THE INVENTION

The present invention contrives to solve the disadvantages of the prior art. The present invention provides a dental mesh for covering a membrane material placed on a bone graft material filling a tooth socket of a patient. The dental mesh includes a mesh and a plurality of attachment needles which are constructed to penetrate into gum tissue to secure the mesh in place.

The object of the invention is to provide a dental mesh for covering a membrane material which is placed on a bone graft material filling the tooth socket. The dental mesh includes a mesh and a plurality of attachment needles. The mesh may be substantially rectangular, having opposite sides across a bone graphite location from one gum tissue to the opposite gum tissue. Each needle has an end having a pointed tip and an opposite end connected to the mesh. The attachment needles are curved such that the pointed tips of the first and second attachment needles face each other or downwardly to respectively penetrate into the opposite gum tissues for securing the mesh to the gum tissues.

The attachment needles may be connected to strands extending from the mesh. The attachment needles may be “J”- shaped, compound curved, or “C”-shaped. Alternatively, they may be “L”-shaped with pointed tips. Furthermore, the mesh may include a plurality of protrusions, which extend and taper downwardly from the mesh in order to penetrate into the membrane for preventing the membrane from moving.

Another object of the invention is to provide a method to install the dental mesh of the present invention. The method includes the steps of: (a) filling a tooth socket with a bone graft material; (b) placing a dental mesh to cover the bone graft material where the dental mesh includes a mesh and first and second attachment needles; (c) lifting a gum tissue and penetrating the first attachment needle into the gum tissue to secure the first attachment needle to the gum tissue; (d) laying the gum tissue down; (e) lifting an opposite gum tissue and penetrating the second attachment needle into the opposite gum tissue to secure the second attachment needle to the gum tissue; and (f) laying the opposite gum tissue down. Laying the opposite gum tissue down generates tension between the gum tissues which helps to secure membrane in place. The tension can be adjusted by adjusting the penetration location of the gum tissue by the attachment needle.

Still another object of the invention is to provide a dental mesh for covering a bone graft material or a membrane. The dental mesh may include a mesh and first and second attachment needles. The mesh has opposite sides which extend to opposite gum tissues respectively, and the first and second attachment needles respectively extend from the opposite sides of the mesh. The first and second attachment needles are curved to penetrate into the opposite gum tissues respectively for securing the mesh to the opposite gum tissues. When the dental mesh directly covers the bone graft material, the mesh, made of membrane material, plays the role of the membrane with the help of other means to prevent invasion of cells not involved in bone formation. Such means is necessary because the mesh is placed on the gum tissues, not thereunder, and thus, gum tissue cells can grow into the bone graft site.

The advantages of the present invention are: (1) the dental mesh of the present invention makes the dental bone graft procedure much easier, convenient, and effective by getting rid of the suturing or stitching process; (2) the dental mesh of the present invention is easy and convenient to install by using the plurality of attachment needles, which is both easier to use and more effective in holding the membrane in place than conventional suturing methods, which require significant skills and have high defect and failure rates; (3) it is easier and more convenient to perform the dental bone graft surgery by using the dental mesh of the present invention; (4) the attachment needles of the dental mesh are easy to attach to and remove from the gum tissues; (5) the dental mesh of the present invention makes controlling or adjusting the tension applied to the membrane easier by simply adjusting the proximity between the gum tissues, compared to suturing gum tissues where such tension adjustment is hard to achieve; (6) the method to install the dental mesh is simple and easy, but effective, especially for adjusting the tension applied to the membrane; (7) using the dental mesh of the present invention, the membrane is well secured in place and less likely to move, slip, or be dislocated or dislodged; (8) the mesh can be laterally tensile and expandable by making the longitudinal strands and lateral strands form an acute angle, and it is easy to adjust the tensile force of the mesh to securely hold the membrane in place; (9) the dental mesh of the present invention is less likely to lead to gum tissue damage than conventional suturing; (10) the dental mesh of the present invention has lower dental bone graft defects or failures; (11) the patient can more comfortably brush his or her teeth and use mouthwash, because the membrane is well secured and there is less discomfort from using the dental mesh of the present invention; and (12) the dental mesh of the present invention has a simple structure, and thus, is easy to manufacture at a low cost.

Although the present invention is briefly summarized, the fuller understanding of the invention can be obtained by the following drawings, detailed description and appended claims.

### BRIEF DESCRIPTION OF THE DRAWINGS

These and other features, aspects and advantages of the present invention will become better understood with reference to the accompanying drawings, wherein:

FIG. 1 shows a schematic view of a tooth;

FIG. 2 shows a schematic view of the dental mesh, covering the membrane and bone graft material, according to the present invention;

FIG. 3 shows another schematic view of the dental mesh, covering the membrane and bone graft material, according to the present invention;

FIG. 4 shows a schematic top view of the dental mesh, covering the membrane and bone graft material, according to the present invention;

FIG. 5 shows a top view of the dental mesh according to one embodiment of the present invention;

FIG. 6A shows an enlarged view of the attachment needle having a “J” shape needle according to one embodiment of the resent invention;

FIG. 6B shows an enlarged view of the “C”-shaped attachment needle according to another embodiment of the present invention;

FIG. 6C shows an enlarged view of the “L”-shaped attachment needle according to still another embodiment of the present invention;

FIG. 7A shows the mesh having one exemplary attachment needle according to one embodiment of the present invention;

FIG. 7B shows the “J” shape attachment needle and the strand before the strand is fixedly attached to the “J” shape attachment needle according to the present invention;

FIG. 7C shows the “J” shape attachment needle and the strand where the strand is fixedly attached to the “J” shape attachment needle according to the present invention;

FIG. 8 shows a schematic side view of the dental mesh, showing the protrusions, according to one embodiment of the present invention;

FIG. 9 shows an enlarged schematic side view of the dental mesh, showing the protrusions, according to one embodiment of the present invention;

FIG. 10 shows the mesh according to one embodiment of the present invention;

FIG. 11 shows the mesh according to another embodiment of the present invention;

FIG. 12 shows the mesh which is rectangle according to the present invention;

FIG. 13 shows the mesh where the two sides are inwardly curved according to the present invention; and

FIG. 14 shows various shapes of surgical needles.

### DETAILED DESCRIPTION OF EMBODIMENTS OF THE INVENTION

Reference will now be made in detail to the preferred embodiments of the present invention, examples of which are illustrated in the accompanying drawings, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention.

Also, as used in the specification including the appended claims, the singular forms “a”, “an”, and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about”, it will be understood that the particular value forms another embodiment.

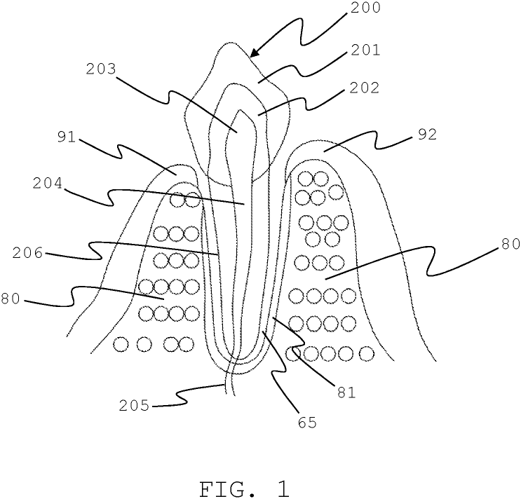

FIG. 1 shows a schematic view of a tooth 200. A tooth 200 is covered with enamel 201, which functions as a protective barrier. Dentin 202, located underneath the enamel 201, extends almost the entire length of the tooth to cover the pulp chamber 203 and pulp canal 204 which contain blood vessels and nerves 205. The cementum 206 covers the root of the tooth 200 whereas the periodontal ligament 81 covers the alveolar bone 80. The periodontal ligament 81 supports the tooth 200 by absorbing the forces of biting and chewing. The gum tissue 91, 92 protects the alveolar bone 80 and covers the neck of the tooth 200. Once a tooth 200 is pulled or removed, a hole called a tooth socket 65 is formed in the alveolar bone 80 where the tooth 200 was located.

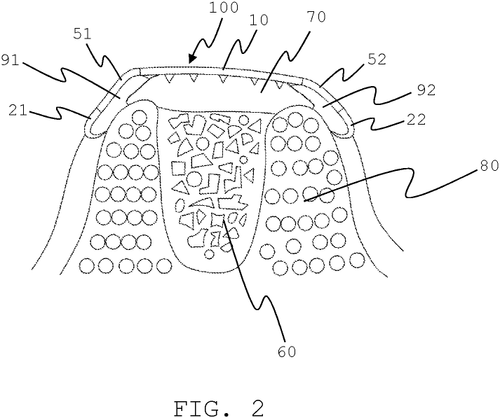

FIGS. 2-4 show schematic views of the dental mesh 100 according to the present invention. A dental bone graft procedure includes filling a bone graft material 60 into the tooth socket 65 and covering the bone graft material 60 with a membrane material 70. Conventional method includes suturing a gum tissue 91, 92 to secure the membrane material 70 in place. The present invention provides a dental mesh 100 to secure the membrane material 70 in place. The dental mesh 100 for covering a membrane material 70 or a bone graft material 60 includes a mesh 10 and first and second attachment needles 21, 22. The mesh 10 has four sides 11, 12, 13, 14, and when the mesh 10 is placed on the membrane material 70, the opposite sides 11, 12 are spaced to extend from one gum tissue 91 to the opposite gum tissue 92. The first and second attachment needles 21, 22 include needles 21, 22, which have an end 31, 32 having a pointed tip and an opposite end 41, 42 connected to the mesh 10.

Preferably, the pointed tips 31, 32 have reverse cutting needles because conventional cutting needles have greater risk of tissue cutout. The pointed tips 31, 32 can be in various shapes, but triangular shape is preferred.

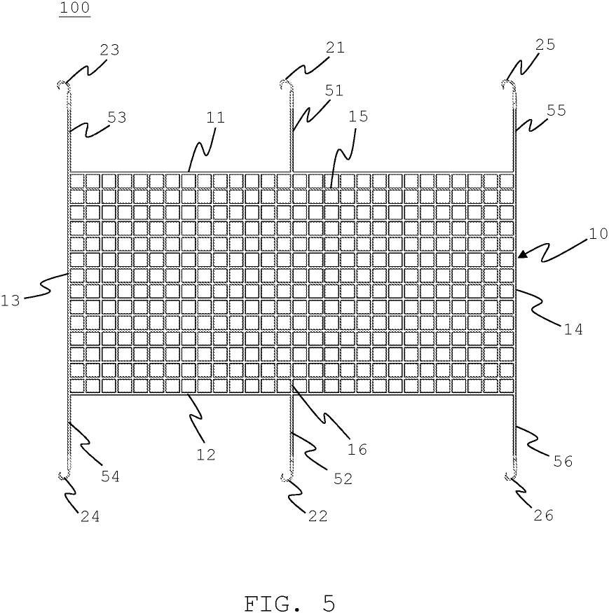

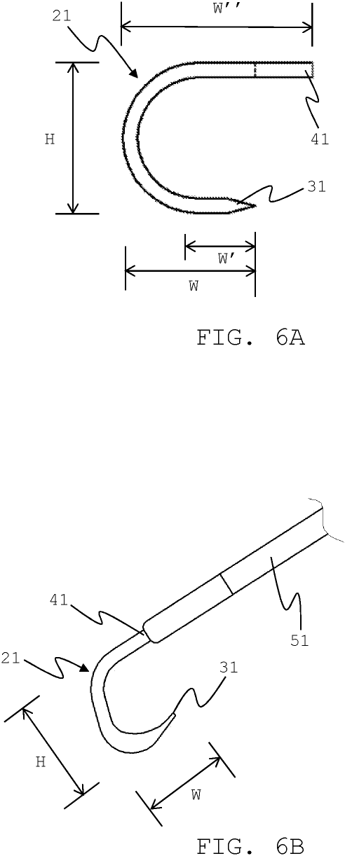

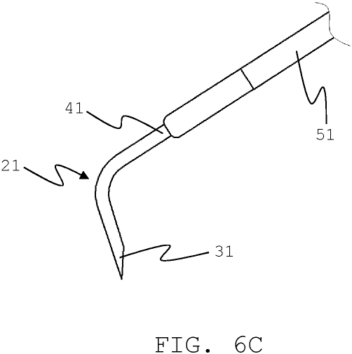

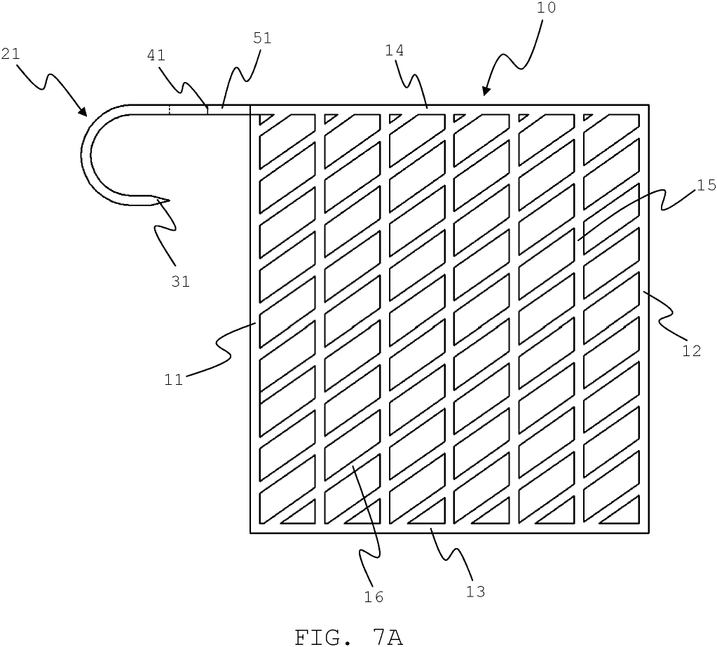

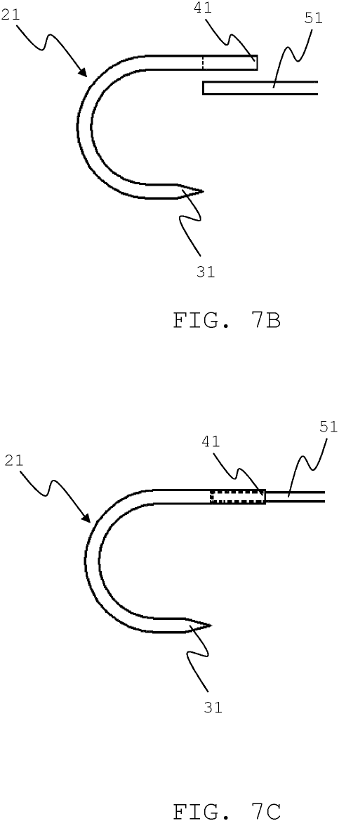

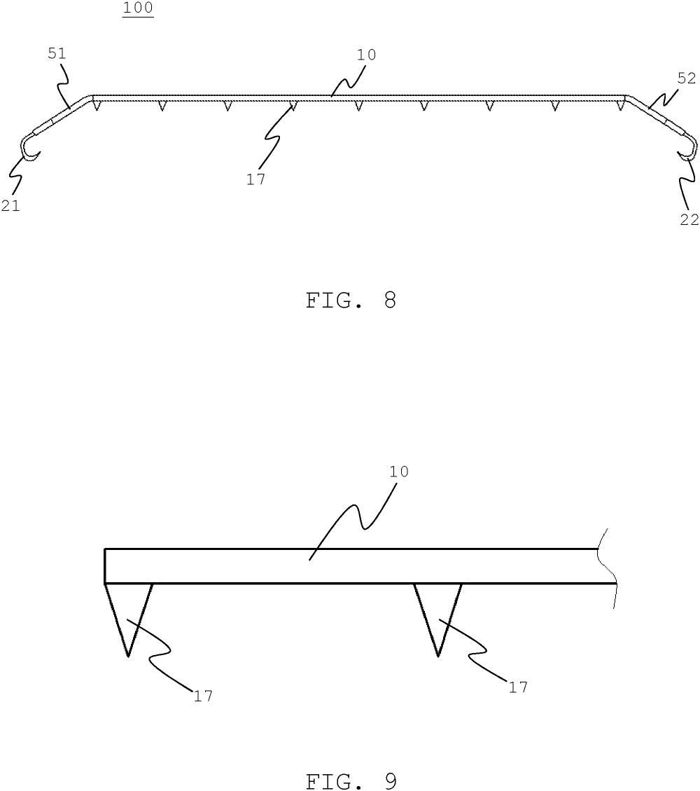

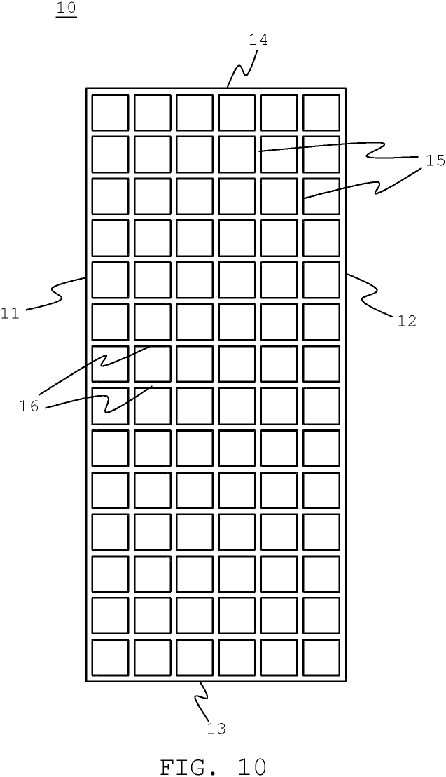

As shown in FIG. 5, the first and second attachment needles 21, 22 (or 23, 24) are respectively attached to the opposite sides 11, 12 of the mesh 10. As shown in FIGS. 6A, 6B, and 6C, the first and second attachment needles 21, 22 are curved such that the pointed tips 31, 32 of the first and second attachment needles 21, 22 face each other or downwardly to respectively penetrate into the opposite gum tissues 91, 92 for securing the mesh 10 to the opposite gum tissues 91, 92. The first and second attachment needles 21, 22 penetrate into the opposite gum tissues 91, 92 to be secured therein by the tension between the first and second attachment needles 21, 22. Here, “curved” includes not a just smoothly bending line such as a “J” or “C”-shaped line but also a sharply bent line such as an “L”-shaped line. Accordingly, the curved attachment needles 21, 22 may be “J”-shaped as in FIG. 6A, “C”-shaped as in FIG. 6B or “L”-shaped as in FIG. 6C. The pointed tips 31, 32 of the “J-shaped or “C”-shaped attachment needles 21, 22 face each other whereas the pointed tips 31, 32 of the “L”-shaped attachment needles 21, 22 face downwardly. FIG. 14 shows various shapes of surgical needles, and preferably, the first and second attachment needles 21, 22 are a “J” shape needle or a compound curved needle where the pointed tips 31, 32 of the first and second attachment needles 21, 22 face each other. Since the pointed tips 31, 32 face each other, instead of downwardly, it is less likely that the pointed tips 31, 32 accidentally pierce into the gum tissue when they are out of the gum tissue. Furthermore, since the pointed tips 31, 32 face laterally, they are well secured in the gum tissues and less likely to get out of the gum tissues.

The mesh 10 may be made of suture material. Suture material may be absorbable or non-absorbable, each of which may be made of natural or synthetic materials. Absorbable suture material includes calgut, chromic catgut, dexon (polyglycolic acid), vicryl (polyglacitin), PDS (polydioxanone), collagen, maxon (polyglyconate), poliglecaprone, etc., and non-absorbable suture material includes silk, linen, cotton, horse/human hair, nylon or ethilon, polyester (Teflon), polypropylene (prolene), stainless steel, aluminium wire, clips, staples, skin tapes, surgical adhesives, etc. The ranges of diameter of the suture material are 0.100~0.149 mm, 0.150~0.199 mm, 0.200~0.249 mm, 0.300~0.349 mm, 0.350~0.399 mm, 0.400~0.499 mm, etc.

Alternatively, the mesh 10 may be made of biocompatible three dimensional print materials such as MED625, ABS-M30i, Vero ContactClear, Biocompatible Digital ABS, etc. MED625 is a flexible clear material with approximate 50% elongation at break and a shore hardness of 75A. ABS-M30i has sufficient strength with biocompatibility and sterilization capability. In addition, Vero ContactClear is transparent and enables rapid manufacturing. These materials or combination of these materials may be used to manufacture the mesh 10.

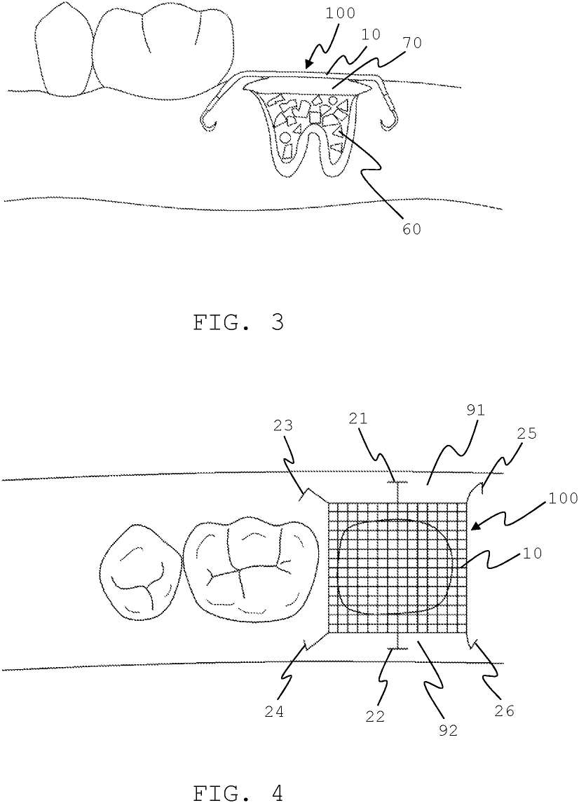

The gum tissues surround the teeth to form a tight seal, functioning as a barrier against bacteria. The opposite gum tissues 91, 92 mean two opposite locations of the gum tissue, surrounding the tooth socket 65, where the first and second attachment needles 21, 22 penetrate into in order to tighten and secure the mesh 10 in place. Preferably, the opposite gum tissues 91, 92 are part of the continuous gum tissues on both sides of the teeth as shown in FIG. 4. FIG. 3 describes the attachment needles 21, 22 which penetrate into gum tissues between the teeth, but this is an illustration only, and correct locations of the attachment needles 21, 22 to penetrate are as described in FIG. 4.

The membrane material 70 is placed in between the gum tissue 91, 92 and the alveolar bone 80 to cover the bone graft material 60 to prevent the invasion of gum cells which grow faster than bone cells. For effective function of the membrane material 70, it has to be securely held in place.

First and second strands 51, 52 respectively extend from the opposite sides 11, 12 of the mesh 10, and the first and second strands 51, 52 are respectively connected to the first and second attachment needles 21, 22. Preferably, the first and second strands 51, 52 constitute a single strand which crosses the mesh as shown in FIG. 5. The opposite ends 41, 42 of the first and second attachment needles 21, 22 may have an eye or swaged end to respectively received the first and second strands 51, 52 therein. The first and second strands 51, 52 are fixedly attached to hollow ends 41, 42 of the first and second attachment needles 21, 22. Preferably, the first and second strands 51, 52 are preferably about 4.0 mm in length, and about 2.0 mm of the strands 51, 52 are received in the hollow ends 41, 42.

The first and second attachment needles 21, 22 may be “J” shaped or compound curved. In one embodiment, the attachment needle 21, 22 is about 3.0 mm in width (W), and about 5.0 mm in height (H). The width (W′) of the pointed tip 31 is about 2.0 mm, and the width (W″) including the hollow end 41 is about 5.0 mm with the hollow depth of about 2.0 mm. In another embodiment, the first and second attachment needles 21, 22 may be “C”-shaped with about 1.5 mm width and about 2.0 mm height. Alternatively, the first and second strands 51, 52 are about 4.0 mm in length, and the first and second attachment needles 21, 22 are “L”-shaped. “L”-shaped attachment needles 21, 22 may look similar to a surgical clip or staple.

The mesh 10 may be comprised of a plurality of longitudinal strands 15 and a plurality of lateral strands 16 as shown in FIGS. 5, 10, or 11, where the longitudinal strands 15 and the lateral strands 16 are fixedly attached to each other at intersecting points. Additionally, the mesh 10 may include a plurality of protrusions 17, which extend and taper downwardly from the intersecting points to enable the protrusion 17 to penetrate into the membrane material 70 for preventing the membrane material 70 from moving. Preferably, the protrusion 17 has the height of about 0.5~1.0 mm and width of about 1.0 mm. The protrusion 17 penetrates into the membrane material 70, but does not pierce through it.

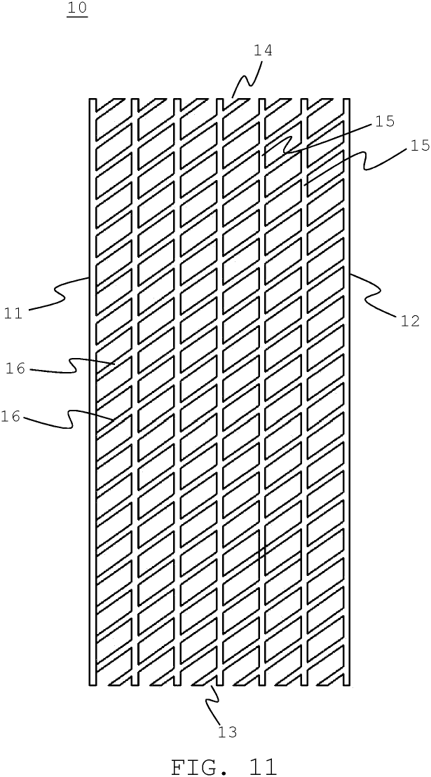

As in FIGS. 5, 10, or 11, the longitudinal strands 15 are parallel to each other and the lateral strands 16 are parallel to each other as well. As in FIGS. 5 or 10, the longitudinal strands 15 are perpendicular to the lateral strands 16, or as in FIG. 11, the longitudinal strands 15 may not be perpendicular to the lateral strands 16. In addition, the first and second attachment needles 21, 22 may be respectively attached to both ends of a lateral strand 16.

The longitudinal strands 15 may be parallel to each other to form a rectangle, and the lateral strands 16 may be parallel to each other to be located about within the rectangle. Preferably, the size of the mesh 10 is about 3 mm by 1.0 cm for a single incisor and about 5 mm by 1.0 cm for a single molar. Preferably, the strands 15, 16 form a grid comprised of a plurality of squares. Each square may be sized in between about 0.1 mm by 0.1 mm and about 0.15 mm by 0.15 mm, with a maximum size of about 1.5 mm by 1.5 mm. Alternatively, as in FIG. 11, the longitudinal strands 15 may not be perpendicular to the lateral strands 16, making the mesh 10 laterally expandable. Since the mesh 10 is laterally expandable and tensile, it is advantageous to generate tension in order to securely hold the membrane material 70 in place.

The longitudinal strands 15 may form a first layer and the lateral strands 16 may form a second layer, and at least one of the first and second layers is in contact with the membrane material 70. The protrusion 17 may extend from either the first layer or the second layer to penetrate into the membrane material 70. Alternatively, the longitudinal strands 15 and the lateral strands 16 may be interwoven each other.

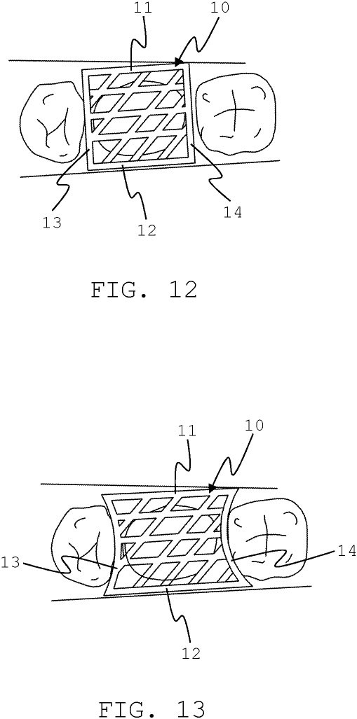

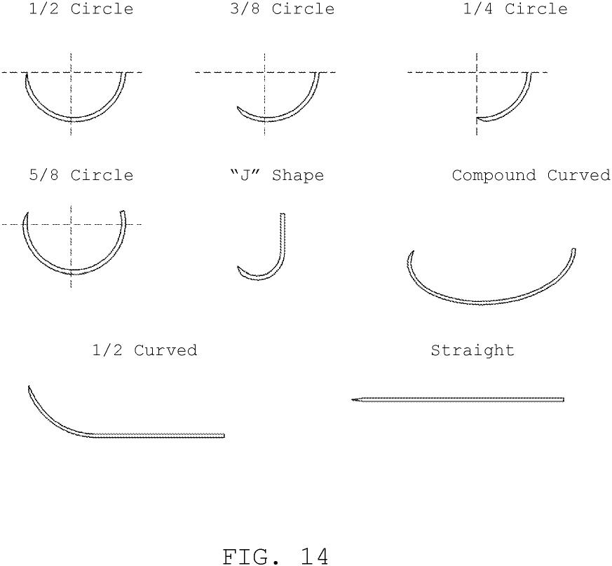

In one embodiment, the mesh 10 has four sides 11, 12, 13, 14 where the mesh 10 is rectangle as in FIG. 12. In another embodiment, the opposite sides 11, 12 are parallel to each other and the other two sides 13, 14 are inwardly curved as in FIG. 13. The inwardly curved sides 13, 14 respectively border adjacent teeth and the membrane material 70 can be better secured by the mesh 10.

The dental mesh 100 of the present invention may further include third and fourth attachment needles 23, 24 where each needle includes an end 33, 34 having a pointed tip and an opposite end 43, 44 connected to the mesh 10. The third and fourth attachment needles 23, 24 are respectively attached to the opposite sides 11, 12 of the mesh 10. The third and fourth attachment needles 23, 24 are curved such that the pointed tips 33, 34 of the third and fourth attachment needles 23, 24 face each other or downwardly to respectively penetrate into the opposite gum tissues 91, 92 for securing the mesh 10 to the opposite gum tissues 91, 92.

Furthermore, the dental mesh 100 of the present invention may further comprising fifth and sixth attachment needles 25, 26 where each needle includes an end 35, 36 having a pointed tip and an opposite end 45, 46 connected to the mesh 10. The fifth and sixth attachment needles 25, 26 are respectively attached to the opposite sides 11, 12 of the mesh 10. The fifth and sixth attachment needles 25, 26 are curved such that the pointed tips 35, 36 of the fifth and sixth attachment needles 25, 26 face each other or downwardly to respectively penetrate into the opposite gum tissues 91, 92 for securing the mesh 10 to the opposite gum tissues 91, 92,

In preferred embodiment, the dental mesh 100 has either four attachment needles 21, 22, 23, 24, or six attachment needles 21, 22, 23, 24, 25, 26. In case of four attachment needles 21, 22, 23, 24, they are respectively connected to the four corners of the rectangular mesh 10. In case of six attachment needles 21, 22, 23, 24, 25, 26, four of them are respectively connected to the four corners of the rectangular mesh 10 and the other two attachment needles are respectively connected to about middle of the opposite sides 11, 12 of the mesh 10.

The method for a dental bone graft according to the present invention includes the steps of: filling a tooth socket 65 with a bone graft material 60; placing a dental mesh 100 to cover the bone graft material 60 wherein the dental mesh 100 comprises a mesh 10 and first and second attachment needles 21, 22; penetrating the first attachment needle 21 into a gum tissue 91 to secure the first attachment needle 21 to the gum tissue 91; lifting an opposite gum tissue 92 and penetrating the second attachment needle 22 into the opposite gum tissue 92 to secure the second attachment needle 22 to the opposite gum tissue 92; and laying the opposite gum tissue 92 down. Furthermore, the method may include the step of placing a membrane material 70 on the bone graft material 60 after the step of filling the tooth socket 65 with the bone graft material 60 where the membrane material 70 is placed between the gum tissue 91 and an alveolar bone 80. Then, the dental mesh 100 is placed on the membrane material 70.

By lifting the gum tissue 92 and penetrating it with the attachment needle 22, a user can adjust the tension between the first and second attachment needles 21, 22 by adjusting the penetration location. If the tension is too strong, the gum tissue may break or bleed, and if too weak, the force applied to the membrane material 70 is not sufficient and the membrane material 70 may move. Thus, the present invention is easy to adjust such tension by lifting the gum tissue 92 to figure out the penetration location and penetrating the gum tissue 92 with the second attachment needle 22. Laying the gum tissue 92 generates tension between the first and second attachment needles 21, 22. Accordingly, the present invention has lower failure or defect rate, compared to conventional sutures.

The method may further comprise the step of adjusting the mesh 10 so that the mesh 10 fully covers the membrane material 70 and pressing the mesh 10 to the membrane material 70 so that a protrusion 17 of the dental mesh 100 penetrates into the membrane material 70. The protrusion 17 extends and tapers downwardly from the mesh 10. Preferably, the protrusion 17 has the height of about 0.5~1.0 mm and width of about 1.0 mm. The protrusion 17 penetrates into the membrane material 70, but does not pierce through it. In addition, the method may further comprise the step of lifting the gum tissue 91 before the step of penetrating the first attachment needle 21 into the gum tissue 91.

The first and second attachment needles 21, 22 may have a “J” shape or a compound curved needle. Alternatively, the first and second attachment needles 21, 22 may be “C”-shaped or “L”-shaped. The angle of the “L” shape may be 90 degrees or less. The mesh 10 may further include first and second strands 51, 52 respectively extending from the opposite sides 11, 12 of the mesh 10 for about 4.0 mm in length.

In still alternative embodiment, the dental mesh 100 for covering a bone graft material 60 or a membrane material 70 according to the present invention includes a rectangular mesh 10, first and second strands 51, 52, and first and second attachment needles 21, 22. The rectangular mesh 10 has opposite sides 11, 12 which are constructed to respectively extend to opposite gum tissues 91, 92. The first and second strands 51, 52 respectively extending from the opposite sides 11, 12 of the rectangular mesh 10, and the first and second attachment needles 21, 22 are respectively attached to the first and second strands 51, 52.

When the dental mesh 100 directly covers the bone graft material 60, the dental mesh 100 plays the role of the membrane with the help of other means to prevent invasion of cells not involved in bone formation. The dental mesh 100 may be made of material, which is used to manufacture a membrane.

The first and second attachment needles 21, 22 are curved to respectively penetrate into the opposite gum tissues 91, 92 for securing the mesh 10 to the opposite gum tissues 91, 92. The first and second attachment needles 21, 22 penetrate into the opposite gum tissues 91, 92 to be secured therein by the tension between the first and second attachment needles 21, 22. Here, “curved” includes not a just smoothly bending line such as a “J”-shaped or “C”-shaped line but also a sharply bent line such as an “L”-shaped line. Accordingly, the curved attachment needles 21, 22 may be “J”-shaped or compound curved as in FIG. 6A, “C”-shaped as in FIG. 6B or “L”-shaped as in FIG. 6C.

The first and second attachment needles 21, 22 may be “C”-shaped with about 1.5 mm width and about 2.0 mm height. Alternatively, the first and second attachment needles 21, 22 may be “L”-shaped. The angle of the “L” shape may be 90 degrees or less. The strands 51, 52 extending from the mesh 10 may be about 4.0 mm in length.

The mesh 10 may further include a plurality of protrusions 17 which extends and tapers downwardly from the mesh 10 to enable the protrusions 17 to penetrate into the membrane material 70 or the bone graft material 60. Such protrusions 17 prevent the membrane material 70 from moving. The protrusion may have the height of about 0.5~1.0 mm and width of about 1.0 mm.

While the invention has been shown and described with reference to different embodiments thereof, it will be appreciated by those skilled in the art that variations in form, detail, compositions and operation may be made without departing from the spirit and scope of the invention as defined by accompanying claims.

What is claimed is:

1. A dental mesh for covering a membrane material or a bone graft material, comprising:
a mesh having opposite sides which are spaced to respectively extend to opposite gum tissues; and
first and second attachment needles, each needle comprising an end having a pointed tip and an opposite end connected to the mesh,
wherein the first and second attachment needles are respectively attached to the opposite sides of the mesh,
wherein the first and second attachment needles are curved such that the pointed tips of the first and second attachment needles face each other or downwardly to respectively penetrate into the opposite gum tissues for securing the mesh to the opposite gum tissues.

2. The dental mesh of claim 1, wherein first and second strands respectively extend from the opposite sides of the mesh, and wherein the first and second strands are respectively connected to the first and second attachment needles.

3. The dental mesh of claim 2, wherein the first and second attachment needles are a “J” shape needle or a compound curved needle wherein the pointed tips of the first and second attachment needles face each other.

4. The dental mesh of claim 1, wherein the mesh is made of suture material.

5. The dental mesh of claim 1, wherein the mesh comprises a plurality of longitudinal strands and a plurality of lateral strands, wherein the longitudinal strands and the lateral strands are fixedly attached to each other at intersecting points.

6. The dental mesh of claim 5, wherein a protrusion extends and tapers downwardly from an intersecting point to enable the protrusion to penetrate into the membrane material.

7. The dental mesh of claim 5, wherein the longitudinal strands are parallel to each other and the lateral strands are parallel to each other,
wherein the longitudinal strands are perpendicular to the lateral strands, and
wherein the first and second attachment needles are respectively attached to both ends of a lateral strand.

8. The dental mesh of claim 5, wherein the longitudinal strands are parallel to each other to form a rectangle, and the lateral strands are parallel to each other to be located about within the rectangle,
wherein the longitudinal strands are not perpendicular to the lateral strands.

9. The dental mesh of claim 1, wherein the mesh has four sides wherein the opposite sides are parallel to each other and the other two sides are inwardly curved.

10. The dental mesh of claim 1, further comprising third and fourth attachment needles, each needle comprising an end having a pointed tip and an opposite end connected to the mesh, wherein the third and fourth attachment needles are respectively attached to the opposite sides of the mesh,
wherein the third and fourth attachment needles are curved such that the pointed tips of the third and fourth attachment needles face each other or downwardly to respectively penetrate into the opposite gum tissues for securing the mesh to the opposite gum tissues.

11. The dental mesh of claim 10, further comprising fifth and sixth attachment needles, each needle comprising an end having a pointed tip and an opposite end connected to the mesh,
wherein the fifth and sixth attachment needles are respectively attached to the opposite sides of the mesh,
wherein the fifth and sixth attachment needles are curved such that the pointed tips of the fifth and sixth attachment needles face each other or downwardly to respectively penetrate into the opposite gum tissues for securing the mesh to the opposite gum tissues,
wherein the mesh is formed into a rectangle,
wherein four attachment needles out for the first, second, third, fourth, fifth, and sixth attachment needles are respectively connected to four corners of the rectangle.

12. A method for a dental bone graft, comprising the steps of:
filling a tooth socket with a bone graft material;
placing a dental mesh to cover the bone graft material wherein the dental mesh comprises a mesh and first and second attachment needles;
penetrating the first attachment needle into a gum tissue to secure the first attachment needle to the gum tissue;
lifting an opposite gum tissue and penetrating the second attachment needle into the opposite gum tissue to secure the second attachment needle to the opposite gum tissue; and
laying the opposite gum tissue down.

13. The method of claim 12, further comprising the step of placing a membrane material on the bone graft material after the step of filling the tooth socket with the bone graft material, wherein the membrane material is placed between the gum tissue and an alveolar bone,
wherein the dental mesh is placed on the membrane material.

14. The method of claim 13, further comprising the step of adjusting the mesh so that the mesh fully covers the membrane material and pressing the mesh to the membrane material so that a protrusion of the dental mesh penetrates into the membrane material, wherein the protrusion extends and tapers downwardly from the mesh.

15. The method of claim 13, further comprising the step of lifting the gum tissue before the step of penetrating the first attachment needle into the gum tissue.

16. The dental mesh of claim 12, wherein the first and second attachment needles are a “J” shape needle or a compound curved needle wherein the pointed tips of the first and second attachment needles face each other.

17. A dental mesh for covering a bone graft material or a membrane material, comprising:
a mesh having four sides of opposite sides and the other two sides wherein the opposite sides are spaced to respectively extend to opposite gum tissues;
first and second strands respectively extending from the opposite sides of the rectangular mesh;
first and second attachment needles, respectively attached to the first and second strands,
wherein the first and second attachment needles are curved to respectively penetrate into the opposite gum tissues for securing the mesh to the opposite gum tissues,
wherein the opposite sides are parallel to each other and the other two sides are either parallel to each other or inwardly curved.

18. The dental mesh of claim 17, wherein the first and second attachment needles are a “J” shape needle or a compound curved needle wherein the pointed tips of the first and second attachment needles face each other.

19. The dental mesh of claim 17, wherein the mesh is made of suture material.

20. The dental mesh of claim 20, wherein a plurality of protrusions extends and tapers downwardly from the mesh to enable the protrusions to penetrate into the membrane material or the bone graft material.
